(19) United States
(12) Patent Application Publication
(10) Pub. No.: US 2011/0097707 A1
(43) Pub. Date: Apr. 28, 2011

(54) RNAi Agents Comprising Universal Nucleobases

(75) Inventors: Muthiah MANOHARAN, Cambridge, MA (US); Kallanthottathil G. RAJEEV, Cambridge, MA (US)

(73) Assignee: ALNYLAM PHARMACEUTICALS, INC., Cambridge, MA (US)

(21) Appl. No.: 12/915,529

(22) Filed: Oct. 29, 2010

Related U.S. Application Data
(63) Continuation of application No. 11/834,140, filed on Aug. 6, 2007, now abandoned.
Application No. 11/834,140 is a continuation-in-part of application No. 11/186,915, filed on Jul. 21, 2005, now Pat. No. 7,579,451.
(60) Provisional application No. 60/589,632, filed on Jul. 21, 2004.
(60) Provisional application No. 60/598,596, filed on Aug. 4, 2004.
(60) Provisional application No. 60/614,111, filed on Sep. 29, 2004.

(51) Int. Cl. C12Q 1/70; C07H 21/00

(52) U.S. Cl. 435/5; 536/25.3; 536/24.5

(57) ABSTRACT

One aspect of the present invention relates to an oligonucleotide agent comprising at least one universal nucleobase. In certain embodiments, the universal nucleobase is difluorotolyl, nitroindolyl, nitropyrrolyl, or nitroimidazolyl. In a preferred embodiment, the universal nucleobase is difluorotolyl. In certain embodiments, the oligonucleotide is double-stranded. In certain embodiments, the oligonucleotide is single-stranded. Another aspect of the present invention relates to a method of altering the expression level of a target in the presence of target sequence polymorphism. In a preferred embodiment, the oligonucleotide agent alters the expression of different alleles of a gene. In another preferred embodiment, the oligonucleotide agent alters the expression level of two or more genes. In another embodiment, the oligonucleotide agent alters the expression level of a viral gene from different strains of the virus. In another embodiment, the oligonucleotide agent alters the expression level of genes from different species.

### RELATED APPLICATIONS

This application is a continuation of U.S. application Ser. No. 11/834,140, filed Aug. 6, 2007, which is a continuation-in-part of U.S. patent application Ser. No. 11/186,915, filed Jul. 21, 2005; which claims the benefit of priority to U.S. Provisional Patent Application Ser. No. 60/589,632, filed Jul. 21, 2004; U.S. Provisional Patent Application Ser. No. 60/598,596, filed Aug. 4, 2004; and U.S. Provisional Patent Application Ser. No. 60/614,111, filed Sep. 29, 2004. The contents of each of these applications is hereby incorporated by reference in its entirety.

### BACKGROUND OF THE INVENTION

Many diseases (e.g., cancers, hematopoietic disorders, endocrine disorders, and immune disorders) arise from the abnormal expression or activity of a particular gene or group of genes. Similarly, disease can result through expression of a mutant form of protein, as well as from expression of viral genes that have been integrated into the genome of their host. The therapeutic benefits of being able to selectively silence these abnormal or foreign genes are obvious.

Oligonucleotide compounds have important therapeutic applications in medicine. Oligonucleotides can be used to silence genes that are responsible for a particular disease. Gene-silencing prevents formation of a protein by inhibiting translation. Importantly, gene-silencing agents are a promising alternative to traditional small, organic compounds that inhibit the function of the protein linked to the disease. siRNA, antisense RNA, and micro-RNA are oligonucleotides that prevent the formation of proteins by gene-silencing.

RNA interference or “RNAi” is a term initially coined by Fire and co-workers to describe the observation that double-stranded RNA (dsRNA) can block gene expression when it is introduced into worms (Fire et al. (1998) Nature 391, 806-811). Short dsRNA directs gene-specific, post-transcriptional silencing in many organisms, including vertebrates, and has provided a new tool for studying gene function. RNAi is mediated by RNA-induced silencing complex (RISC), a sequence-specific, multicomponent nuclease that destroys messenger RNAs homologous to the silencing trigger. RISC is known to contain short RNAs (approximately 22 nucleotides) derived from the double-stranded RNA trigger, but the protein components of this activity remained unknown.

siRNA compounds are promising agents for a variety of diagnostic and therapeutic purposes. siRNA compounds can be used to identify the function of a gene. In addition, siRNA compounds offer enormous potential as a new type of pharmaceutical agent which acts by silencing disease-causing genes. Research is currently underway to develop interference RNA therapeutic agents for the treatment of many diseases including central-nervous-system diseases, inflammatory diseases, metabolic disorders, oncology, infectious diseases, and ocular disease.

siRNA has been shown to be extremely effective as a potential anti-viral therapeutic with numerous published examples appearing recently. siRNA molecules directed against targets in the viral genome dramatically reduce viral titers by orders of magnitude in animal models of influenza (Ge et. al., Proc. Natl. Acd. Sci. USA, 101:8676-8681 (2004); Tompkins et. al., Proc. Natl. Acd. Sci. USA, 101:8682-8686 (2004); Thomas et. al., Expert Opin. Biol. Ther. 5:495-505 (2005)), respiratory synctial virus (RSV) (Bitko et. al., Nat. Med. 11:50-55 (2005)), hepatitis B virus (HBV) (Morrissey et. al., Nat. Biotechnol. 23:1002-1007 (2005)), hepatitis C virus (Kapadia, Proc. Natl. Acad. Sci. USA, 100:2014-2018 (2003); Wilson et. al., Proc. Natl. Acad. Sci. USA, 100:2783-2788 (2003)) and SARS coronavirus (Li et. al., Nat. Med. 11:944-951 (2005)).

Antisense methodology is the complementary hybridization of relatively short oligonucleotides to mRNA or DNA such that the normal, essential functions, such as protein synthesis, of these intracellular nucleic acids are disrupted. Hybridization is the sequence-specific hydrogen bonding via Watson-Crick base pairs of oligonucleotides to RNA or single-stranded DNA. Such base pairs are said to be complementary to one another.

The naturally-occurring events that alter the expression level of the target sequence, discussed by Cohen (Oligonucleotides: Antisense Inhibitors of Gene Expression, CRC Press, Inc., 1989, Boca Raton, Fla.) are thought to be of two types. The first, hybridization arrest, describes the terminating event in which the oligonucleotide inhibitor binds to the target nucleic acid and thus prevents, by simple steric hindrance, the binding of essential proteins, most often ribosomes, to the nucleic acid. Methyl phosphonate oligonucleotides (Miller et al. (1987) Anti-Cancer Drug Design, 2:117-128), and α-anomer oligonucleotides are the two most extensively studied antisense agents which are thought to disrupt nucleic acid function by hybridization arrest.

Another means by which antisense oligonucleotides alter the expression level of target sequences is by hybridization to a target mRNA, followed by enzymatic cleavage of the targeted RNA by intracellular RNase H. A 2′-deoxyribofuranosyl oligonucleotide or oligonucleotide analog hybridizes with the targeted RNA and this duplex activates the RNase H enzyme to cleave the RNA strand, thus destroying the normal function of the RNA. Phosphorothioate oligonucleotides are the most prominent example of an antisense agent that operates by this type of antisense terminating event.

Despite advances in siRNA, antisense and other oligonucleotide based technologies, one of the major hurdles is overcoming the degeneracy in the genetic code. This degeneracy in the genetic code frequently causes sequence ambiguities and cases where sequence data is available ambiguities can still remain due to polymorphic or species-dependent sequence differences. Particularly, viral sequences are prone to mutation and highly conserved targets may vary among viral strands or related viral families. Therefore, to overcome target-sequence mutation and diversity for any given gene, it would be of value to have a universal base oligonucleotide probe that is capable of selective hybridization even in the presence of polymorphisms. The oligonucleotides of the invention comprising a universal nucleobase fulfill this need by reducing the need for absolute complementarity between the oligonucoleotide probe and the target, thus providing a tool to create oligonucleotide agents that are broader in scope.

### SUMMARY OF THE INVENTION

The present invention provides oligonucleotide compounds comprising a universal nucleobase, and methods for their preparation. The oligonucleotides of the invention include single-stranded and double-stranded oligonucleotides. These oligonucleotide agents can modify gene expression, either inhibiting or up-regulating, by targeting and binding to a nucleic acid, e.g., a pre-mRNA, an mRNA, a microRNA (miRNA), a mi-RNA precursor (pre-miRNA), or DNA, or to a protein. Oligonucleotide agents of the invention include modified siRNA, microRNA, antisense RNA, decoy RNA, DNA, and aptamers. The oligonucleotides of the invention can alter the expression level of target sequences through a RISC pathway dependent or independent mechanism.

Degeneracy in the genetic code frequently causes sequence ambiguities and cases where sequence data is available ambiguities can still remain due to polymorphic or species-dependent sequence differences. Particularly, viral sequences are prone to mutation and highly conserved targets may vary among viral strands or related viral families. Therefore, to overcome target-sequence mutation and diversity for any given gene, it would be of value to have a universal base oligonucleotide agent that is capable of selective hybridization even in the presence of polymorphisms. Use of universal bases may reduce the need for absolute complementarity between the oligonucleotide probe and the target thus providing a tool to create oligonucleotide agents that are broader in scope.

One aspect of the present invention relates to a method of cleaving or silencing a target in the presence of target sequence polymorphism. The method comprises providing an oligonucleotide comprising a universal nucleobase, wherein the oligonucleotide is able to hybridize with the target even in the presence of target polymorphism.

In one preferred embodiment, the oligonucleotide agent cleaves or silences two or more different genes, e.g., a viral and non viral gene. It is preferred that the non-viral gene be a host gene required by the virus.

In another embodiment, the oligonucleotide agent cleaves or silences a viral gene from different strains of the virus. In yet another embodiment of the invention, the gene targeted by the oligonucleotide is from different mutations in the same viral gene.

In another embodiment, the oligonucleotide agent cleaves or silences a target from different species. It is preferred that target represent the same gene in the different species.

In another embodiment, the oligonucleotide agent cleaves or silences a target representing different microRNAs. The microRNAs can be from same family or different families.

This application incorporates all cited references, patent, and patent applications by reference in their entirety for all purposes.

### BRIEF DESCRIPTION OF FIGURES

FIG. 1 depicts a procedure for solid-phase oligonucleotide synthesis.

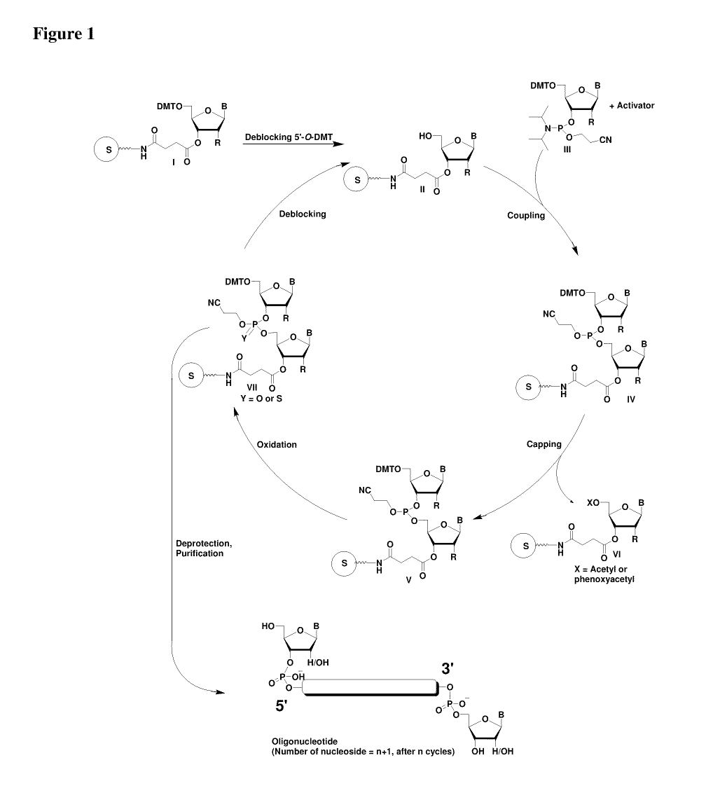

FIG. 2 depicts a procedure for the synthesis of a nitroindole nucleoside. Note: a) MeOH-conc. H₂SO₄, RT, 16 h. b) KOH/18-crown-6/THF/DCBnC1, RT, 16 h. c) HOAc-HBr/CH₂Cl₂, 0-RT, 4 h. d) NaH/CH₃CN, RT, 4-6 h. e) BC1₃/CH₂Cl₂, −78 to −45° C., 4 h. f) MDTrCl/pyridine, DMAP, RT, 16 h. g) AgNO₃-pyridine/THF, RT, TBDMSCl, RT, 4 h. h) (i-Pr)₂NP(Cl)—OCH₂CH₂CN/CH₂Cl₂/DMAP, 4 h, RT.

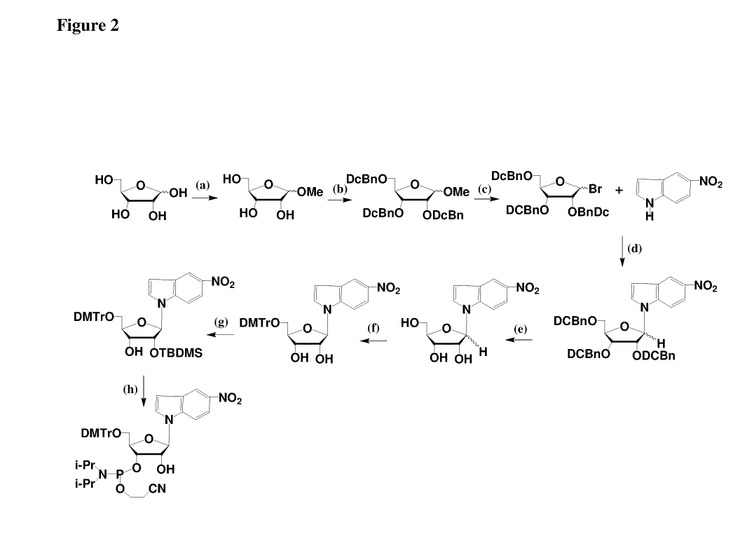

FIG. 3 depicts certain preferred nucleosides of the invention.

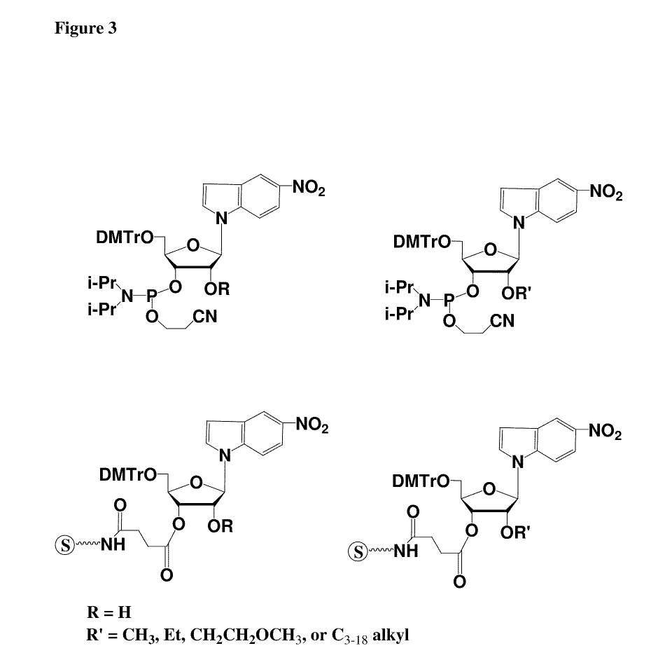

FIG. 4 depicts schematic of sequence alignment of target genes for design of complimentary siRNAs incorporating universal bases.

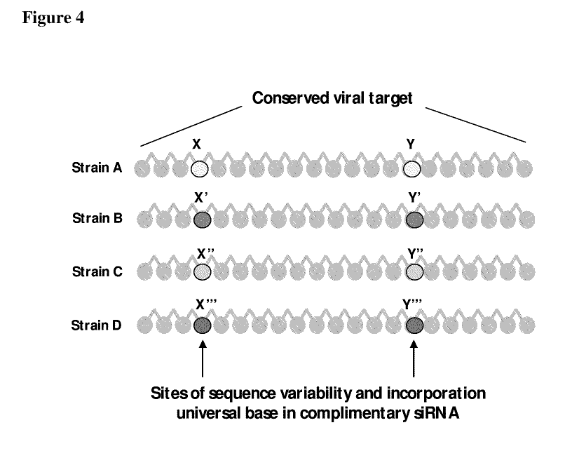

FIG. 5 depicts a schematic of 5-nitroindole comprising siRNAs and mismatch comprising siRNAs. See Exemplification (Table 2) for sequence details for each duplex.

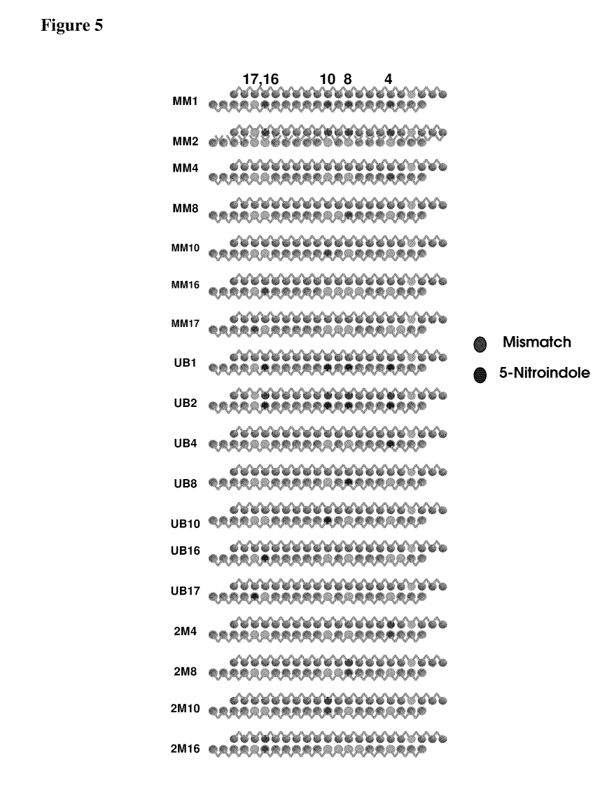

FIG. 6 depicts ELISA based in vitro viral inhibition by modified siRNAs containing 5-nitroindole universal base with respect to unmodified control duplex DP-1685 and mismatch control siRNA duplexes. See Exemplification (Table 2) for sequence details of each duplex.

FIG. 7 depicts influenza A NP gene silencing, in dual luciferase gene silencing assay, by modified siRNAs containing 5-nitroindole universal base with respect to unmodified control duplex DP-1685 and mismatch control siRNA duplexes. See exemplification (Table 2) for sequence details of each duplex.

FIG. 8 depicts ELISA based in vitro viral inhibition by modified siRNAs containing 2,4-difluorotoluyl or inosine base with respect to unmodified control duplex DP-7611 (H1N1) or CU/AG (H3N2) and mismatch control siRNA duplexes. See Exemplification (Table 3) for sequence details of each duplex.

FIG. 9 depicts influenza A NP gene silencing, in dual luciferase gene silencing assay, by modified siRNAs containing 5-nitroindole universal base with respect to unmodified control duplex DP-1685 and mismatch control siRNA duplexes. See Exemplification (Table 3) for sequence details of each duplex.

FIG. 10 depicts a schematic of 2,4-difluorotoluyl comprising siRNA duplexes and ELISA based in vitro viral inhibition by modified siRNAs containing 2,4-difluorotoluyl universal base with respect to unmodified control duplex DP-1685 and mismatch control siRNA duplexes. See Exemplification (Table 3) for sequence details of each duplex.

FIG. 11 depicts influenza A NP gene silencing, in dual luciferase gene silencing assay, by modified siRNAs containing 2,4-difluorotoluyl universal base with respect to unmodified control duplex DP-1685 and mismatch control siRNA duplexes. See Exemplification (Table 3) for sequence details of each duplex.

### DETAILED DESCRIPTION OF THE INVENTION

Degeneracy in the genetic code frequently causes sequence ambiguities and cases where sequence data is available ambiguities can still remain due to polymorphic or species-dependent sequence differences. Particularly, viral sequences are prone to mutation and highly conserved targets may vary among viral strands or related viral families. Therefore, to overcome target-sequence mutation and diversity for any given gene, it would be of value to have a universal base oligonucleotide agent that is capable of selective hybridization even in the presence of polymorphisms. Use of universal bases may reduce the need for absolute complementarity between the oligonucleotide probe and the target thus providing a tool to create oligonucleotide agents that are broader in scope.

In the context of the invention, “hybridization” means hydrogen bonding, which may be Watson-Crick, Hoogsteen or reversed Hoogsteen hydrogen bonding, between complementary nucleosides or nucleotides. For example, adenine and thymine are complementary nucleobases which pair through the formation of hydrogen bonds. “Complementary,” as used herein, refers to the capacity for base-pairing between two nucleotides. The base-pairing between the two nucleobases may or may not involve hydrogen bonding. For example, the universal nucleoside 2,4-difluorotolune is considered to base pair with adenine without the formation of hydrogen bonds between the two nucleobases, while 8-aza-7-deazaadenine-N⁸-(2′-deoxyribonucleoside) I is a universal base that base pairs with all four natural nucleosides through hydrogen bonding between the nucleobases. As used herein, if a nucleoside at a certain position of an oligonucleotide is capable base-pairing with a nucleoside at the opposite position in a target DNA or RNA molecule, then the oligonucleotide and the DNA or RNA are considered to be complementary to each other at that position. Thus, “specifically hybridizable” and “complementary” are terms which are used to indicate a sufficient degree of complementarity or base pairing such that stable and specific binding occurs between the oligonucleotide and the DNA or RNA target. It is understood in the art that an oligonucleotide need not be 100% complementary to its target DNA sequence to be specifically hybridizable. An oligonucleotide is specifically hybridizable when binding of the oligonucleotide to the target DNA or RNA molecule interferes with the normal function of the target DNA or RNA to cause a decrease or loss of function, and there is a sufficient degree of complementarity to avoid non-specific binding of the oligonucleotide to non-target sequences under conditions in which specific binding is desired, i.e., under physiological conditions in the case of in vivo assays or therapeutic treatment, or in the case of in vitro assays, under conditions in which the assays are performed.

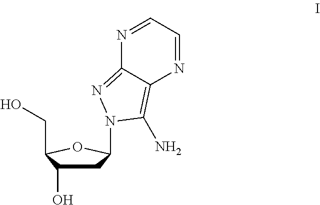

As used herein, a universal base is any modified, unmodified, naturally occurring or non-naturally occurring nucleobase that can pair with all of the four naturally occurring bases without substantially affecting the melting behavior, recognition by intracellular enzymes or activity of the oligonucleotide duplex.

Difluorotoluene nucleoside II is a nonpolar, nucleoside isostere developed as a useful tool in probing the active sites of DNA polymerase enzymes and DNA repair enzymes. See Schweitzer, B. A.; Kool, E. T. J. Org. Chem. 1994, 59, 7238; Schweitzer, B. A.; Kool, E. T. J. Am. Chem. Soc. 1995, 117, 1863; Moran, S. Ren, R. X.-F. Rumney, S.; Kool, E. T. J. Am. Chem. Soc. 1997, 119, 2056; Guckian, K. M.; Kool, E. T. Angew. Chem. Int. Ed. Engl. 1997, 36, 2825; and Mattray, T. J.; Kool, E. T. J. Am. Chem. Soc. 1998, 120, 6191. For additional information see Fire, A.; Xu, S.; Montgomery, M. K.; Kostas, S. A.; Driver, S. E.; Mello, C. C. Nature, 1998, 391, 806; Elbashir, S. M.; Harborth, J.; Lendeckel, W.; Yalcin, A.; Weber, K.; Tuschl, T. Nature, 2001, 411, 494; McManus, M. T. Sharp, P. A. Nature Reviews Genetics, 2002, 3, 737; Hannon, G. J. Nature, 2002, 418, 244; and Roychowdhury, A.; Illangkoon, H.; Hendrickson, C. L.; Benner, S. A. Org. Lett. 2004, 6, 489.

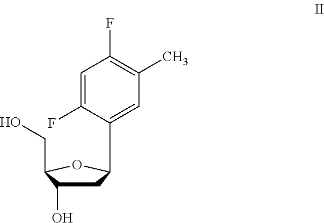

Difluorotolyl is a non-natural nucleobase that functions as a universal base. In contrast to the stabilizing, hydrogen-bonding interactions associated with naturally occurring nucleobases, it is postulated that oligonucleotide duplexes containing universal nucleobases are stabilized solely by stacking interactions. The absence of significant hydrogen-bonding interactions with universal nucleobases obviates the specificity for a specific complementary base. Difluorotolyl is an isostere of the natural nucleobase thymine. But unlike thymine, difluorotolyl shows no appreciable selectivity for any of the natural bases. Other aromatic compounds that function as universal bases and are amenable to the present invention are 4-fluoro-6-methylbenzimidazole and 4-methylbenzimidazole. In addition, the relatively hydrophobic isocarbostyrilyl derivatives 3-methyl isocarbostyrilyl, 5-methyl isocarbostyrilyl, and 3-methyl-7-propynyl isocarbostyrilyl are universal bases which cause only slight destabilization of oligonucleotide duplexes compared to the oligonucleotide sequence containing only natural bases. Other non-natural nucleobases contemplated in the present invention include 7-azaindolyl, 6-methyl-7-azaindolyl, imidizopyridinyl, 9-methyl-imidizopyridinyl, pyrrolopyrizinyl, isocarbostyrilyl, 7-propynyl isocarbostyrilyl, propynyl-7-azaindolyl, 2,4,5-trimethylphenyl, 4-methylindolyl, 4,6-dimethylindolyl, phenyl, napthalenyl, anthracenyl, phenanthracenyl, pyrenyl, stilbenyl, tetracenyl, pentacenyl, and structural derivates thereof. For a more detailed discussion, including synthetic procedures, of difluorotolyl, 4-fluoro-6-methylbenzimidazole, 4-methylbenzimidazole, and other non-natural bases mentioned above, see: Schweitzer et al., J. Org. Chem., 59:7238-7242 (1994); Berger et al., Nucleic Acids Research, 28(15):2911-2914 (2000); Moran et al., J. Am. Chem. Soc., 119:2056-2057 (1997); Morales et al., J. Am. Chem. Soc., 121:2323-2324 (1999); Guckian et al., J. Am. Chem. Soc., 118:8182-8183 (1996); Morales et al., J. Am. Chem. Soc., 122(6):1001-1007 (2000); McMinn et al., J. Am. Chem. Soc., 121:11585-11586 (1999); Guckian et al., J. Org. Chem., 63:9652-9656 (1998); Moran et al., Proc. Natl. Acad. Sci., 94:10506-10511 (1997); Das et al., J. Chem. Soc., Perkin Trans., 1:197-206 (2002); Shibata et al., J. Chem. Soc., Perkin Trans., 1:1605-1611 (2001); Wu et al., J. Am. Chem. Soc., 122(32):7621-7632 (2000); O'Neill et al., J. Org. Chem., 67:5869-5875 (2002); Chaudhuri et al., J. Am. Chem. Soc., 117:10434-10442 (1995); and U.S. Pat. No. 6,218,108.

Nitropyrrolyl and nitroindolyl are non-natural nucleobases that are also considered to belong to the class of compounds known as universal bases. It is postulated that oligonucleotide duplexes containing 3-nitropyrrolyl nucleobases are stabilized solely by stacking interactions. The absence of significant hydrogen-bonding interactions with nitropyrrolyl nucleobases obviates the specificity for a specific complementary base. In addition, various reports confirm that 4-, 5- and 6-nitroindolyl display very little specificity for the four natural bases. Interestingly, an oligonucleotide duplex containing 5-nitroindolyl was more stable than the corresponding oligonucleotides containing 4-nitroindolyl and 6-nitroindolyl. Procedures for the preparation of 1-(2′-O-methyl-β-D-ribofuranosyl)-5-nitroindole are described in Gaubert, G.; Wengel, J. Tetrahedron Letters 2004, 45, 5629. Other universal bases amenable to the present invention include hypoxanthinyl, isoinosinyl, 2-aza-inosinyl, 7-deaza-inosinyl, nitroimidazolyl, nitropyrazolyl, nitrobenzimidazolyl, nitroindazolyl, aminoindolyl, pyrrolopyrimidinyl, and structural derivatives thereof. For a more detailed discussion, including synthetic procedures, of nitropyrrolyl, nitroindolyl, and other universal bases mentioned above see Vallone et al., Nucleic Acids Research, 27(17):3589-3596 (1999); Loakes et al., J. Mol. Bio., 270:426-436 (1997); Loakes et al., Nucleic Acids Research, 22(20):4039-4043 (1994); Oliver et al., Organic Letters, Vol. 3(13):1977-1980 (2001); Amosova et al., Nucleic Acids Research, 25(10):1930-1934 (1997); Loakes et al., Nucleic Acids Research, 29(12):2437-2447 (2001); Bergstrom et al., J. Am. Chem. Soc., 117:1201-1209 (1995); Franchetti et al., Biorg. Med. Chem. Lett. 11:67-69 (2001); and Nair et al., Nucelosides, Nucleotides & Nucleic Acids, 20(4-7):735-738 (2001).

The modified oligonucleotides of the present invention overcome degenrecy of target sequence by being less selective in pairing with juxtaposing natural bases. In certain embodiments, the universal base is in complementary position to the ambiguous nucleobase position of the target sequences. In certain embodiments, the universal nucleobase is difluorotolyl, nitroindolyl, nitropyrrolyl, or nitroimidazolyl. In certain embodiments, the universal nucleobase is nitroindolyl. In a preferred embodiment, the universal nucleobase is difluorotolyl.

In the context of this invention, siRNA comprises double-stranded oligonucleotides, wherein the term “oligonucleotide” refers to an oligomer or polymer of ribonucleic acid or deoxyribonucleic acid. This term includes oligonucleotides composed of naturally-occurring nucleobases, sugars and covalent intersugar (backbone) linkages as well as modified or non-natural oligonucleotides having non-naturally-occurring portions which function similarly. Such modified or substituted oligonucleotides are often preferred over native forms because of desirable properties such as, for example, enhanced cellular uptake, enhanced binding to target and increased stability in the presence of nucleases. The oligonucleotides of the present invention preferably comprise from about 5 to about 50 nucleosides. It is more preferred that such oligonucleotides comprise from about 8 to about 30 nucleosides, with 15 to 25 nucleosides being particularly preferred.

It is preferred that the first and second strands be chosen such that the siRNA includes a single strand or unpaired region at one or both ends of the molecule. Thus, siRNA agent contains first and second strands, preferably paired to contain an overhang, e.g., one or two 5′ or 3′ overhangs but preferably a 3′-overhang of 2-3 nucleotides. Most embodiments will have a 3′ overhang. The overhangs can be result of one strand being longer than the other, or the result of two strands of the same length being staggered. The 5′ ends are preferably phosphorylated. Preferably the siRNA is 21 nucleotides in length, and the duplex region of the siRNA is 19 nucleotides.

The single-stranded oligonucleotide agents featured in the invention include antisense nucleic acids. An “antisense” nucleic acid includes a nucleotide sequence that is complementary to a “sense” nucleic acid encoding a gene expression product, e.g., complementary to the coding strand of a double-stranded cDNA molecule or complementary to an RNA sequence, e.g., a pre-mRNA, mRNA, miRNA, or pre-miRNA. Accordingly, an antisense nucleic acid can form hydrogen bonds with a sense nucleic acid target. The single-stranded oligonucleotide compounds of the invention preferably comprise from about 10 to 25 nucleosides (e.g., 11, 12, 13, 14, 15, 16, 18, 19, 20, 21, 22, 23, or 24 nucleotides in length).

While not wishing to be bound by theory, an oligonucleotide agent may act by one or more of a number of mechanisms, including a cleavage-dependent or cleavage-independent mechanism. A cleavage-based mechanism can be RNAse H dependent and/or can include RISC complex function. Cleavage-independent mechanisms include occupancy-based translational arrest, such as is mediated by miRNAs, or binding of the oligonucleotide agent to a protein, as do aptamers. Oligonucleotide agents may also be used to alter the expression of genes by changing the choice of the splice site in a pre-mRNA. Inhibition of splicing can also result in degradation of the improperly processed message, thus down-regulating gene expression. Kole and colleagues (Sierakowska, et al. Proc. Natl. Acad. Sci. USA, 1996, 93:12840-12844) showed that 2′-O-Me phosphorothioate oligonucleotides could correct aberrant beta-globin splicing in a cellular system. Fully modified 2′-methoxyethyl oligonucleotides and peptide nucleic acids (PNAs) were able to redirect splicing of IL-5 receptor-a pre-mRNA (Karras et al., Mol. Pharmacol. 2000, 58:380-387; Karras, et al., Biochemistry 2001, 40:7853-7859).

Oligonucleotide agents discussed include otherwise unmodified RNA and DNA as well as RNA and DNA that have been modified. Examples of modified RNA and DNA include modificiations to improve efficacy and polymers of nucleoside surrogates. Unmodified RNA refers to a molecule in which the components of the nucleic acid, namely sugars, bases, and phosphate moieties, are the same or essentially the same as that which occur in nature, preferably as occur naturally in the human body. The literature has referred to rare or unusual, but naturally occurring, RNAs as modified RNAs. See Limbach et al. Nucleic Acids Res. 1994, 22, 2183-2196. Such rare or unusual RNAs, often termed modified RNAs, are typically the result of a post-transcriptional modification and are within the scope of the term unmodified RNA as used herein. Modified RNA as used herein refers to a molecule in which one or more of the components of the nucleic acid, namely sugars, bases, and phosphate moieties, are different from that which occur in nature, preferably different from that which occurs in the human body. While they are referred to as “modified RNAs” they will of course, because of the modification, include molecules that are not, strictly speaking, RNAs. Nucleoside surrogates are molecules in which the ribophosphate backbone is replaced with a non-ribophosphate construct that allows the bases to the presented in the correct spatial relationship such that hybridization is substantially similar to what is seen with a ribophosphate backbone, e.g., non-charged mimics of the ribophosphate backbone. A nucleotide subunit in which the sugar of the subunit has been so replaced is referred to herein as a sugar replacement modification subunit (SRMS). The SRMS may be the 5′- or 3′-terminal subunit of the oligonucleotide agent and located adjacent to two or more unmodified or modified ribonucleotides. Alternatively, the SRMS may occupy an internal position located adjacent to one or more unmodified or modified ribonucleotides. More than one SRMS may be present in an oligonucleotide agent. Preferred positions for inclusion of a SRMS tethered to a moiety (e.g., a lipophilic moiety such as cholesterol) are at the 3′-terminus, the 5′-terminus, or at an internal position.

The oligonucleotide compounds of the invention can be prepared using solution-phase or solid-phase organic synthesis. Organic synthesis offers the advantage that the oligonucleotide strands comprising non-natural or modified nucleotides can be easily prepared. Any other means for such synthesis known in the art may additionally or alternatively be employed. It is also known to use similar techniques to prepare other oligonucleotides, such as the phosphorothioates, phosphorodithioates and alkylated derivatives. The double-stranded oligonucleotide compounds of the invention comprising non-natural nucleobases and optionally non-natural sugar moieties may be prepared using a two-step procedure. First, the individual strands of the double-stranded molecule are prepared separately. Then, the component strands are annealed.

Teachings regarding the synthesis of particular modified oligonucleotides may be found in the following U.S. patents or pending patent applications: U.S. Pat. Nos. 5,138,045 and 5,218,105, drawn to polyamine conjugated oligonucleotides; U.S. Pat. No. 5,212,295, drawn to monomers for the preparation of oligonucleotides having chiral phosphorus linkages; U.S. Pat. Nos. 5,378,825 and 5,541,307, drawn to oligonucleotides having modified backbones; U.S. Pat. No. 5,386,023, drawn to backbone-modified oligonucleotides and the preparation thereof through reductive coupling; U.S. Pat. No. 5,457,191, drawn to modified nucleobases based on the 3-deazapurine ring system and methods of synthesis thereof; U.S. Pat. No. 5,459,255, drawn to modified nucleobases based on N-2 substituted purines; U.S. Pat. No. 5,521,302, drawn to processes for preparing oligonucleotides having chiral phosphorus linkages; U.S. Pat. No. 5,539,082, drawn to peptide nucleic acids; U.S. Pat. No. 5,554,746, drawn to oligonucleotides having .beta.-lactam backbones; U.S. Pat. No. 5,571,902, drawn to methods and materials for the synthesis of oligonucleotides; U.S. Pat. No. 5,578,718, drawn to nucleosides having alkylthio groups, wherein such groups may be used as linkers to other moieties attached at any of a variety of positions of the nucleoside; U.S. Pat. Nos. 5,587,361 and 5,599,797, drawn to oligonucleotides having phosphorothioate linkages of high chiral purity; U.S. Pat. No. 5,506,351, drawn to processes for the preparation of 2′-O-alkyl guanosine and related compounds, including 2,6-diaminopurine compounds; U.S. Pat. No. 5,587,469, drawn to oligonucleotides having N-2 substituted purines; U.S. Pat. No. 5,587,470, drawn to oligonucleotides having 3-deazapurines; U.S. Pat. No. 5,223,168, and U.S. Pat. No. 5,608,046, both drawn to conjugated 4′-desmethyl nucleoside analogs; U.S. Pat. Nos. 5,602,240, and 5,610,289, drawn to backbone-modified oligonucleotide analogs; and U.S. Pat. Nos. 6,262,241, and 5,459,255, drawn to, inter alia, methods of synthesizing 2′-fluoro-oligonucleotides.

It is not necessary for all positions in a given compound to be uniformly modified, and in fact more than one type of modification may be incorporated in a single oligonucleotide compound or even in a single nucleotide thereof.

One aspect of the present invention relates to a method of cleaving or silencing a target in the presence of target sequence polymorphism. The method comprises providing an oligonucleotide comprising a universal nucleobase, wherein the oligonucleotide is able to hybridize with the target even in the presence of target polymorphism. According to such a method, the polymorphic target sequences are aligned to obtain a consensus target sequence. The oligonucleotide comprising universal nucleobase(s) at positions complementary to variable positions in the consensus target sequence is then prepared and administered.

In one preferred embodiment, the oligonucleotide agent cleaves or silences two or more different genes, e.g., a viral and non viral gene. It is preferred that the non-viral gene be a host gene required by the virus.

In another embodiment, the oligonucleotide agent cleaves or silences a viral gene from different strains of the virus. In yet another embodiment of the invention, the gene targeted by the oligonucleotide is from different mutations in the same viral gene.

In another embodiment, the oligonucleotide agent cleaves or silences a target from different species. It is preferred that target represent the same gene in the different species.

In another embodiment, the oligonucleotide agent cleaves or silences a target representing different microRNAs. The microRNAs can be from same family or different families.

Specific examples of preferred modified oligonucleotides envisioned for use in the oligonucleotides of the present invention include oligonucleotides containing modified backbones or non-natural internucleoside linkages. As defined here, oligonucleotides having modified backbones or internucleoside linkages include those that retain a phosphorus atom in the backbone and those that do not have a phosphorus atom in the backbone. For the purposes the invention, modified oligonucleotides that do not have a phosphorus atom in their intersugar backbone can also be considered to be oligonucleosides.

Representative United States patents that teach the preparation of the phosphorus atom-containing inter-nucleotide linkages include, but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; 5,625,050; and 5,697,248, each of which is herein incorporated by reference.

Representative United States patents that teach the preparation modified internucleoside linkages or backbones that do not include a phosphorus atom therein (i.e., oligonucleosides) include, but are not limited to, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439, each of which is herein incorporated by reference.

In other preferred oligonucleotide mimetics, both the sugar and the internucleoside linkage, i.e., the backbone, of the nucleoside units are replaced with novel groups. The nucleobase units are maintained for hybridization with an appropriate nucleic acid target compound. One such oligonucleotide, an oligonucleotide mimetic, that has been shown to have excellent hybridization properties, is referred to as a peptide nucleic acid (PNA). In PNA compounds, the sugar-backbone of an oligonucleotide is replaced with an amide-containing backbone, in particular an aminoethylglycine backbone. The nucleobases are retained and are bound directly or indirectly to atoms of the amide portion of the backbone. Representative United States patents that teach the preparation of PNA compounds include, but are not limited to, U.S. Pat. Nos. 5,539,082; 5,714,331; and 5,719,262, each of which is herein incorporated by reference. Further teaching of PNA compounds can be found in Nielsen et al., Science, 1991, 254, 1497.

The oligonucleotides employed in the oligonucleotides of the present invention may additionally comprise nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C), and uracil (U). Modified nucleobases include other synthetic and natural nucleobases, such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine.

Further nucleobases include those disclosed in U.S. Pat. No. 3,687,808, those disclosed in the Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B., ed., CRC Press, 1993. Certain of these nucleobases are particularly useful for increasing the binding affinity of the oligonucleotides of the invention. These include 5-substituted pyrimidines, 6-azapyrimidines and N-2, N-6 and O-6 substituted purines, including 2-aminopropyladenine, 5-propynyluracil and 5-propynylcytosine. 5-Methylcytosine substitutions have been shown to increase nucleic acid duplex stability by 0.6-1.2° C. (Id., pages 276-278) and are presently preferred base substitutions, even more particularly when combined with 2′-methoxyethyl sugar modifications.

Representative United States patents that teach the preparation of certain of the above-noted modified nucleobases as well as other modified nucleobases include, but are not limited to, the above noted U.S. Pat. No.3,687,808, as well as U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,681,941; and 5,808,027; all of which are hereby incorporated by reference.

The oligonucleotides employed in the oligonucleotides of the present invention may additionally or alternatively comprise one or more substituted sugar moieties. Preferred oligonucleotides comprise one of the following at the 2′ position: OH; F; O—, S—, or N-alkyl, O—, S—, or N-alkenyl, or O, S— or N-alkynyl, wherein the alkyl, alkenyl and alkynyl may be substituted or unsubstituted C₁ to C₁₀ alkyl or C₂ to C₁₀ alkenyl and alkynyl. Particularly preferred are O[(CH₂)ₙO]ₘCH₃, O(CH₂)ₙOCH₃, O(CH₂)ₙNH₂, O(CH₂)ₙCH₃, O(CH₂)ₙONH₂, and O(CH₂)ₙON[(CH₂)ₙCH₃)]₂, where n and m are from 1 to about 10. Other preferred oligonucleotides comprise one of the following at the 2′ position: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂ CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. A preferred modification includes 2′-methoxyethoxy [2′-O—CH₂CH₂OCH₃, also known as 2′-O-(2-methoxyethyl) or 2′-MOE] (Martin et al., Helv. Chim. Acta, 1995, 78, 486), i.e., an alkoxyalkoxy group. A further preferred modification includes 2′-dimethylaminooxyethoxy, i.e., a O(CH₂)₂ON(CH₃)₂ group, also known as 2′-DMAOE, as described in U.S. Pat. No. 6,127,533, the contents of which are incorporated by reference.

Other preferred modifications include 2′-methoxy (2′-O—CH₃), 2′-aminopropoxy (2′-OCH₂CH₂CH₂NH₂) and 2′-fluoro (2′-F). Similar modifications may also be made at other positions on the oligonucleotide, particularly the 3′ position of the sugar on the 3′ terminal nucleotide or in 2′-5′ linked oligonucleotides.

As used herein, the term “sugar substituent group” or “2′-substituent group” includes groups attached to the 2′-position of the ribofuranosyl moiety with or without an oxygen atom. Sugar substituent groups include, but are not limited to, fluoro, O-alkyl, O-alkylamino, O-alkylalkoxy, protected O-alkylamino, O-alkylaminoalkyl, O-alkyl imidazole and polyethers of the formula (O-alkyl)ₘ, wherein m is 1 to about 10. Preferred among these polyethers are linear and cyclic polyethylene glycols (PEGs), and (PEG)-containing groups, such as crown ethers and those which are disclosed by Ouchi et al. (Drug Design and Discovery 1992, 9:93); Ravasio et al. (J. Org. Chem. 1991, 56:4329); and Delgardo et. al. (Critical Reviews in Therapeutic Drug Carrier Systems 1992, 9:249), each of which is hereby incorporated by reference in its entirety. Further sugar modifications are disclosed by Cook (Anti-Cancer Drug Design, 1991, 6:585-607). Fluoro, O-alkyl, O-alkylamino, O-alkyl imidazole, O-alkylaminoalkyl, and alkyl amino substitution is described in U.S. Pat. No. 6,166,197, entitled “Oligomeric Compounds having Pyrimidine Nucleotide(s) with 2′ and 5′ Substitutions,” hereby incorporated by reference in its entirety.

Additional sugar substituent groups amenable to the present invention include 2′-SR and 2′-NR₂ groups, wherein each R is, independently, hydrogen, a protecting group or substituted or unsubstituted alkyl, alkenyl, or alkynyl. 2′-SR Nucleosides are disclosed in U.S. Pat. No. 5,670,633, hereby incorporated by reference in its entirety. The incorporation of 2′-SR monomer synthons is disclosed by Hamm et al. (J. Org. Chem., 1997, 62:3415-3420). 2′-NR nucleosides are disclosed by Goettingen, M., J. Org. Chem., 1996, 61, 6273-6281; and Polushin et al., Tetrahedron Lett., 1996, 37, 3227-3230.

In certain instances, the ribose sugar moiety that naturally occurs in nucleosides is replaced with a hexose sugar, polycyclic heteroalkyl ring, or cyclohexenyl group. In certain instances, the hexose sugar is an allose, altrose, glucose, mannose, gulose, idose, galactose, talose, or a derivative thereof. In a preferred embodiment, the hexose is a D-hexose. In a preferred embodiment, the hexose sugar is glucose or mannose. In certain instances, the polycyclic heteroalkyl group is a bicyclic ring containing one oxygen atom in the ring. In certain instances, the polycyclic heteroalkyl group is a bicyclo[2.2.1]heptane, a bicyclo[3.2.1]octane, or a bicyclo[3.3.1]nonane. In certain instances, the sugar moiety is represented by A′ or A″, wherein Z¹ and Z² each are independently O or S and A² is a nucleobase, e.g., a natural nucleobase, a non-natural nucleobase, a modified nucleobase or a universal nucleobase.

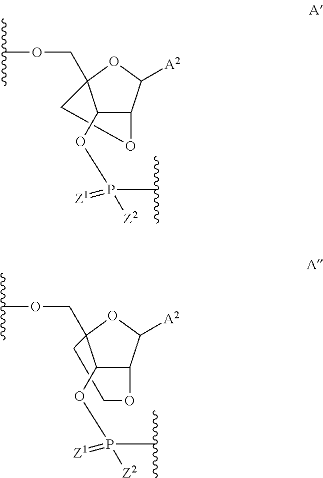

Representative United States patents that teach the preparation of such modified sugars structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,0531 5,639,873; 5,646,265; 5,658,873; 5,670,633; 5,700,920; and 5,859,221, all of which are hereby incorporated by reference.

A wide variety of entities can be tethered to the oligonucleotide agent. A ligand tethered to an oligonucleotide agent can have a favorable effect on the agent. For example, the ligand can improve stability, hybridization thermodynamics with a target nucleic acid, targeting to a particular tissue or cell-type, or cell permeability, e.g., by an endocytosis-dependent or —independent mechanism. Ligands and associated modifications can also increase sequence specificity and consequently decrease off-site targeting. Preferred moieties are ligands, which are coupled, preferably covalently, either directly or indirectly via an intervening tether, to the SRMS carrier. In preferred embodiments, the ligand is attached to the carrier via an intervening tether.

The ligand can be attached at the 3′-terminus, the 5′-terminus, or internally. The ligand can be attached to an SRMS, e.g., a 4-hydroxyprolinol-based SRMS at the 3′-terminus, the 5′-terminus, or at an internal linkage. The attachment can be direct or through a tethering molecule. The ligand can be attached to just one strand or both strands of a double stranded oligonucleotide agent. In certain instances, the oligonucleotide may incorporate more that one ligand, wherein the ligands may all be the same or all different or a combination thereof.

In certain instances, the oligonucleotide may be modified by a non-ligand group. A number of non-ligand molecules have been conjugated to oligonucleotides in order to enhance the activity, cellular distribution or cellular uptake of the oligonucleotide, and procedures for performing such conjugations are available in the scientific literature. Such non-ligand moieties have included lipid moieties, such as cholesterol (Letsinger et al., Proc. Natl. Acad. Sci. USA, 1989, 86:6553), cholic acid (Manoharan et al., Bioorg. Med. Chem. Lett., 1994, 4:1053), a thioether, e.g., hexyl-S-tritylthiol (Manoharan et al., Ann. N.Y. Acad. Sci., 1992, 660:306; Manoharan et al., Bioorg. Med. Chem. Let., 1993, 3:2765), a thiocholesterol (Oberhauser et al., Nucl. Acids Res., 1992, 20:533), an aliphatic chain, e.g., dodecandiol or undecyl residues (Saison-Behmoaras et al., EMBO J., 1991, 10:111; Kabanov et al., FEBS Lett., 1990, 259:327; Svinarchuk et al., Biochimie, 1993, 75:49), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36:3651; Shea et al., Nucl. Acids Res., 1990, 18:3777), a polyamine or a polyethylene glycol chain (Manoharan et al., Nucleosides & Nucleotides, 1995, 14:969), or adamantane acetic acid (Manoharan et al., Tetrahedron Lett., 1995, 36:3651), a palmityl moiety (Mishra et al., Biochim. Biophys. Acta, 1995, 1264:229), or an octadecylamine or hexylamino-carbonyl-oxycholesterol moiety (Crooke et al., J. Pharmacol. Exp. Ther., 1996, 277:923).

Representative United States patents that teach the preparation of such oligonucleotide conjugates include, but are not limited to, U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928; and 5,688,941, each of which is herein incorporated by reference.

Importantly, each of these approaches may be used for the synthesis of oligonucleotides comprising a universal nucleobase.

### Exemplification

The invention now being generally described, it will be more readily understood by reference to the following examples, which are included merely for purposes of illustration of certain aspects and embodiments of the present invention, and are not intended to limit the invention.

### Example 1

### General Procedures for Oligonucleotide Synthesis, Purification, and Analysis

### Synthesis

The RNA molecules (see Table 1, Example 12) can be synthesized on a 394 ABI machine using the standard 93 step cycle written by the manufacturer with modifications to a few wait steps as described below. The monomers can be RNA phosphoramidites with fast protecting groups (5′-O-dimethoxytrityl N6-phenoxyacetyl-2′-O-t-butyldimethylsilyladenosine -3′-O-N,N′-diisopropyl-cyanoethylphosphoramidite, 5′-O-dimethoxytrityl-N4-acetyl-2′-O-t -butyldimethylsilylcytidine-3′-O-N,N′-diisopropyl-2-cyanoethylphosphoramidite, 5′-O-dimethoxytrityl-N2-p-isopropylphenoxyacetyl-2′-O-t-butyldimethylsilylguanosine -3′-O-N,N′-diisopropyl-2-cyanoethylphosphoramidite, and 5′-O-dimethoxytrityl-2′-O-t -butyldimethylsilyluridine-3′-O-N,N′-diisopropyl-2-cyanoethylphosphoramidite from Pierce Nucleic Acids Technologies. 2′-O-Me amidites can be obtained from Glen Research. Amidites are used at a concentration of 0.15M in acetonitrile (CH₃CN) and a coupling time of 12-15 min. The activator is 5-(ethylthio)-1H-tetrazole (0.25M), for the PO-oxidation Iodine/Water/Pyridine can be used and for PS-oxidation, 2% Beaucage reagent (Iyer et al., J. Am. Chem. Soc., 1990, 112, 1253) in anhydrous acetonitrile can be used. The sulphurization time is about 6 min.

### Deprotection-I (Nucleobase Deprotection)

After completion of synthesis the support is transferred to a screw cap vial (VWR Cat # 20170-229) or screw caps RNase free microfuge tube. The oligonucleotide is cleaved from the support with simultaneous deprotection of base and phosphate groups with 1.0 mL of a mixture of ethanolic ammonia [ammonia: ethanol (3:1)] for 15 h at 55° C. The vial is cooled briefly on ice and then the ethanolic ammonia mixture is transferred to a new microfuge tube. The CPG is washed with 2×0.1 mL portions of RNase free deionised water. Combine washings, cool over a dry ice bath for 10 min and subsequently dry in speed vac.

### Deprotection-II (Removal of 2′ TBDMS Group)

The white residue obtained is resuspended in 400 μL of triethylamine, triethylamine trihydrofluoride (TEA.3HF) and NMP (4:3:7) and heated at 50° C. for overnight to remove the tert-butyldimethylsilyl (TBDMS) groups at the 2′ position (Wincott et al., Nucleic Acids Res., 1995, 23, 2677). The reaction is then quenched with 400 μL of isopropoxytrimethylsilane (iPrOMe₃Si, purchase from Aldrich) and further incubate on the heating block leaving the caps open for 10 min; (This causes the volatile isopropxytrimethylsilylfluoride adduct to vaporize). The residual quenching reagent is removed by drying in a speed vac. Added 1.5 mL of 3% triethylamine in diethyl ether and pelleted by centrifuging. The supernatant is pipetted out without disturbing the pellet and the pellet is dried in speed vac. The crude RNA is obtained as a white fluffy material in the microfuge tube.

### Quantitation of Crude Oligomer or Raw Analysis

Samples are dissolved in RNase free deionied water (1.0 mL) and quantitated as follows: Blanking is first performed with water alone (1 mL) 20 μL of sample and 980 μL of water are mixed well in a microfuge tube, transferred to cuvette and absorbance reading obtained at 260 nm. The crude material is dried down and stored at −20° C.

### Purification of Oligomers (PAGE Purification)

PAGE purification of oligomers synthesized is performed as reported by Sambrook et al. (Molecular Cloning: a Laboratory Manual, Second Edition , Cold Spring Harbor Laboratory Press, Cold Spring Harbor, N.Y., 1989). The 12% denaturing gel is prepared for purification of unmodified and modified oligoribonucleotides. Take 120 mL Concentrate+105 mL Diluents+25 mL Buffer (National Diagnostics) then add 50 μL TEMED and 1.5 mL 10% APS. Pour the gel and leave it for ½ h to polymerize. Suspended the RNA in 20 μL water and 80 μL formamide. Load the gel tracking dye on left lane followed by the sample slowly on to the gel. Run the gel on 1× TBE buffer at 36 W for 4-6 h. Once run is completed, Transfer the gel on to preparative TLC plates and see under UV light. Cut the bands. Soak and crushed in Water. Leave in shaker for overnight. Remove the eluent, Dry in speed vac.

### Desalting of Purified Oligomer

The purified dry oligomer is then desalted using Sephadex G-25 M (Amersham Biosciences). The cartridge is conditioned with 10 mL of RNase free deionised water thrice. Finally, the purified oligomer is dissolved in 2.5 mL RNasefree water and passed through the cartridge with very slow drop wise elution. The salt free oligomer is eluted with 3.5 mL of RNase free water directly into a screw cap vial.

### Analysis (Capillary Gel Electrophoresis (CGE) and Electrospray LC/MS)

Approximately 0.10 OD of oligomer is first dried down, then redissolved in water (50 μL) and then pipetted in special vials for CGE and LC/MS analysis.

TABLE 1
2,4-Diflurotluyl (Q10), 5-Nitroindole (Q12)
and Inosine (I) containing oligonucleotides
for constituting siRNAs comprising
modified/unnatural bases(s)

| Seq ID | Sequence (5′-3′) |
|---|---|
| 1 | GGA UCU UAU UUC UUC GGA GdTdT |
| 2 | GGA ACU UUU UUG UUC CGA GdTdT |
| 3 | GGA UCU UAU UUC UUC CGA GdTdT |
| 4 | GGA UCU UAU UUG UUC GGA GdTdT |
| 5 | GGA UCU UAU AUC UUC GGA GdTdT |
| 6 | CUC CGA AGA AAU AAG AUC CdTdT |
| 7 | CUC GGA AGA AAU AAG AUC CdTdT |
| 8 | CUC CGA ACA AAU AAG AUC CdTdT |
| 9 | CUC CGA AGA UAU AAG AUC CdTdT |
| 10 | CUC CGA AGA AAU AAG UUC CdTdT |
| 11 | CUC CGA AGA AAU AAG AAC CdTdT |
| 12 | CUC Q₁₂GA AQ₁₂A Q₁₂AU AAG Q₁₂UC CdTdT |
| 13 | CUC Q₁₂GA AGA AAU AAG AUC CdTdT |
| 14 | CUC CGA AQ₁₂A AAU AAG AUC CdTdT |
| 15 | CUC CGA AGA Q₁₂AU AAG AUC CdTdT |
| 16 | CUC CGA AGA AAU AAG Q₁₂UC CdTdT |
| 17 | CUC CGA AGA AAU AAG AQ₁₂C CdTdT |
| 18 | CUC UGA ACA UAU AAG UUC CdTdT |
| 19 | CUC Q₁₀GA AQ₁₀A Q₁₀AU AAG Q₁₀UC CdTdT |
| 20 | CUC Q₁₀GA AGA AAU AAG AUC CdTdT |
| 21 | CUC CGA AQ₁₀A AAU AAG AUC CdTdT |
| 22 | CUC CGA AGA Q₁₀AU AAG AUC CdTdT |
| 23 | CUC CGA AGA AAU AAG Q₁₀UC CdTdT |
| 24 | CUC CGA AGA AAU AAG AQ₁₀C CdTdT |
| 25 | CGA UCG UGC CUU CCU UUG AdT*dT |
| 26 | CGA UCG UGC CQ₁₂U CQ₁₂U UUG AdT*dT |
| 27 | CGA UCG UGC CQ₁₂U CCU UUG AdT*dT |
| 28 | CGA UCG UGC CIU CIU UUG AdT*dT |
| 29 | UCA AAG GAA GGC ACG AUC GdT*dT |
| 30 | UCA AAQ₁₂ GAQ₁₂ GGC ACG AU GdT*dT |
| 31 | UCA AAG GAQ₁₂ GGC ACG AUC GdT*dT |
| 32 | UCA AAI GAI GGC ACG AU CGdT*dT |
| 33 | CGA UCG UGC CCU CUU UUG AdT*dT |
| 34 | UCA AAA GAG GGC ACG AUC GdT*dT |
| 35 | CGA UCG UGC CCU CCU UUG AdT*dT |
| 36 | UCA AAG GAG GGC ACG AUC GdT*dT |
| 37 | GGA ACU UAU UUC UUC GGA GdTdT |

In Table 1 above, * indicates a phosphorothioate
linkage; Q₁₀ indicates a 2,4-difluorotoluyl
(2,4-difluorotoluene); Q₁₂ indicates a
5-nitroindolyl (5-nitroindole) and I indicates
inosine.

### Example 2

### Efficacy of Universal Base Containing siRNA Duplexes by ELISA Assay

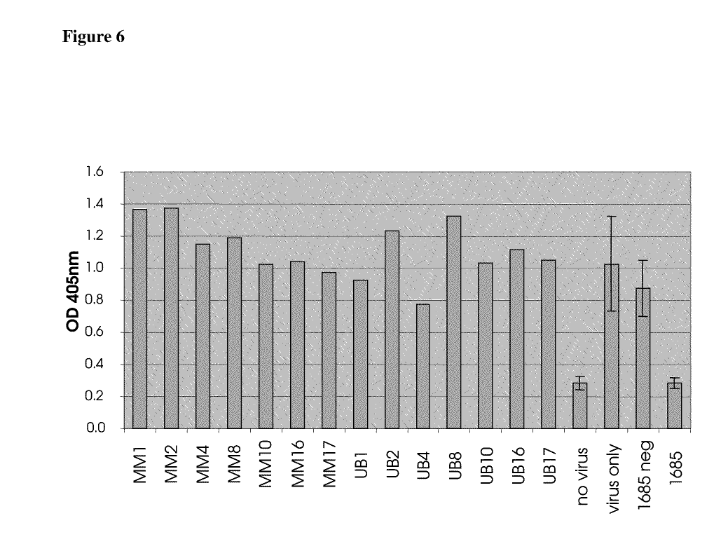

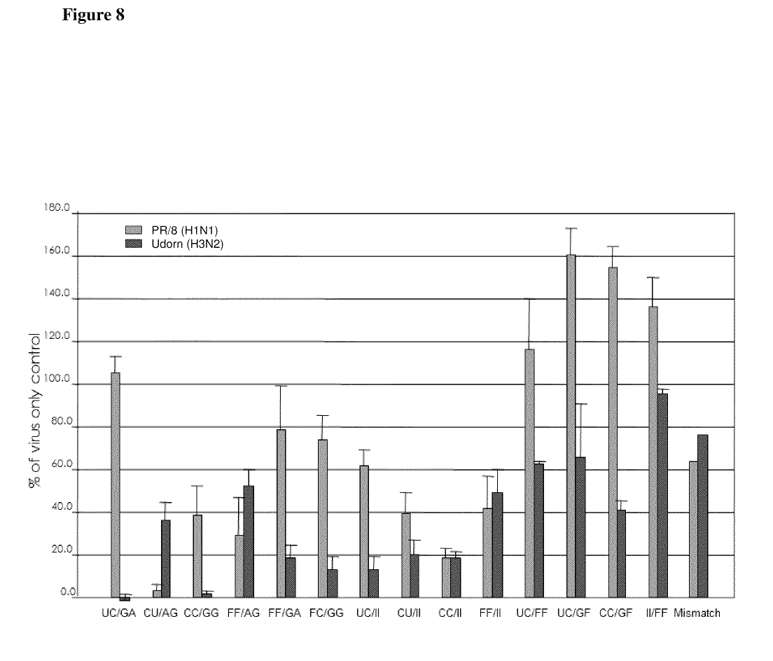

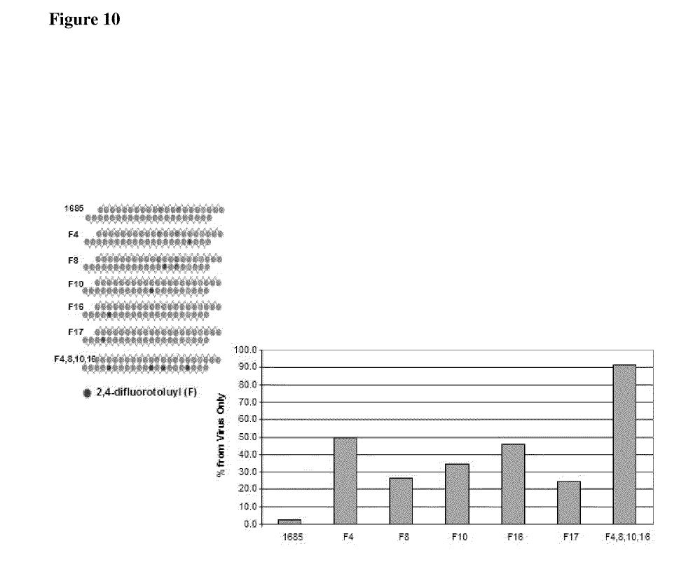

In vitro activity of siRNAs can be determined using an ELISA assay. MDCK or Vero cells are plated in 96-well plate and transfected with the virus targeting siRNAs. The siRNA transfections are performed using Lipofectamin 2000 (Invitrogen) with 35 nM of the duplex. After 14 h, the siRNA transfection medium is removed, and virus (PR/8 (H1N1) or Udorn (H3N2)), in MEM medium, is added to the cells. After 48 h, cells are analyzed for influenza A nucleoprotein using the ELISA assay with biotinylated anti-influenza A monoclonal antibody MAB8258B (Chemicon), AP-conjugated streptavidin (Vector Laboratories) and pNPP substrate. See FIGS. 6, 8 and 10.

### Example 3

### Efficacy of Universal Base Containing siRNA Duplexes by Dual Luciferase Reporter Gene Silencing Assay

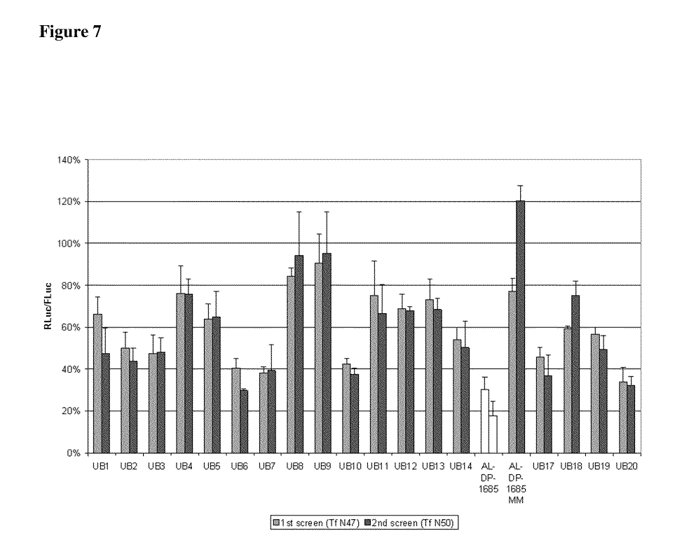

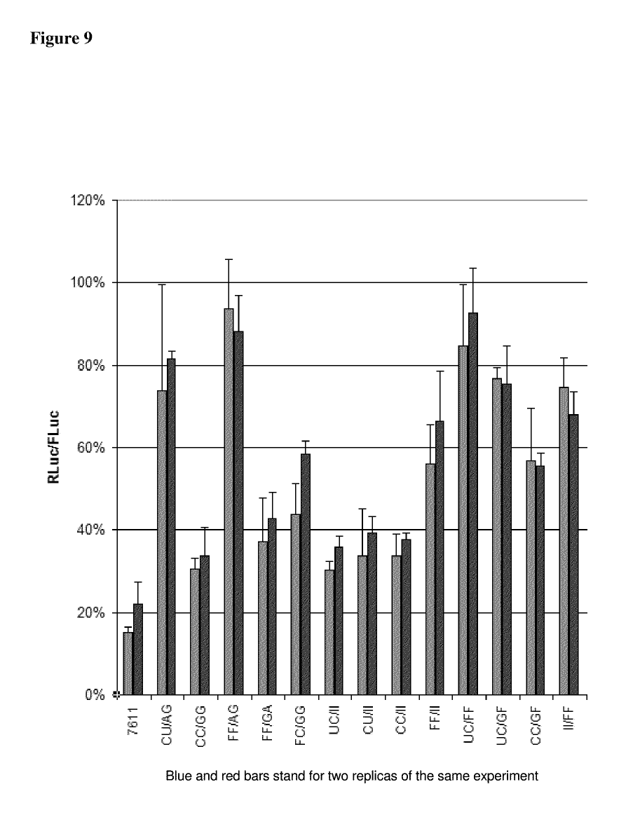

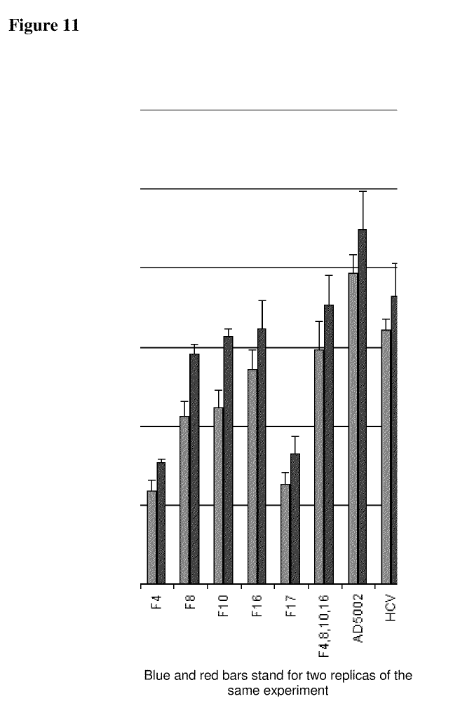

In vitro activity of siRNAs can be determined using a high-throughput 96-well plate format luciferase reporter gene silencing assay. Consensus sequence of the influenza NP gene is subcloned between stop-codon and polyA-signal of Renilla-Luciferase gene of psiCheck-2 Vector (Promega, Mannheim, Germany) via XhoI and NotI sites. Cos-7 cells are first transfected with plasmid encoding Influenza NP gene. DNA transfections are performed using Lipofectamine 2000 (Invitrogen) and 50 ng/well of the plasmid. After 4 h, cells are transfected with influenza NP gene targeting siRNAs at 50 nM concentration using Lipofectamine 2000. After 24 h, cells are analyzed for both firefly and renilla luciferase expression using a plate luminometer (Victor-Light 1420 Luminescence Counter, PerkinElmer, Boston, Mass.) and the Dual-Glo Luciferase Assay kit (Promega). Fireflylrenilla luciferase expression ratios are used to determine percent gene silencing relative to mock-treated (no siRNA) controls. See FIGS. 7, 9 and 11.

### Example 4

### siRNA Duplex Preparation

The two strands of the duplex were arrayed into PCR tubes or plates (VWR, West Chester, Pa.) in phosphate buffered saline to give a final concentration of 20 μM duplex (Table 2). Annealing was performed employing a thermal cycler (ABI PRISM 7000, Applied Biosystems, Foster City, Calif.) capable of accommodating the PCR tubes or plates. The oligoribonucleotides were held at 90° C. for two minutes and 37° C. for one hour prior to use in assays.

TABLE 2
5-Nitroindole comprising siRNA duplexes.

| Duplex | Seq ID | Sequence | Modification |
|---|---|---|---|
| 1685 | 1 | GGA UCU UAU UUC UUC GGA G dTdT | Positive control siRNA |
| | 6 | dTdT CCU AGA AUA AAG AAG CCU C | |
| MM1 | 1 | GGA UCU UAU UUC UUC GGA G dTdT | One each of G:U, C:C, U:U |
| | 18 | dTdT CCU UGA AUA UAC AAG UCU C | and U:U mismatch pairs |
| MM2 | 2 | GGA ACU UUU UUG UUC CGA G dTdT | One each of A:A, U:U, G:G |
| | 6 | dTdT CCU AGA AUA AAG AAG CCU C | and C:C mismatch pairs |
| MM4 | 1 | GGA UCU UAU UUC UUC GGA G dTdT | Single G:G mismatch pair |
| | 7 | dTdT CCU AGA AUA AAG AAG GCU C | |
| MM8 | 1 | GGA UCU UAU UUC UUC GGA G dTdT | Single C:C mismatch pair |
| | 8 | dTdT CCU AGA AUA AAC AAG CCU C | |
| MM10 | 1 | GGA UCU UAU UUC UUC GGA G dTdT | Single U:U mismatch pair |
| | 9 | dTdT CCU AGA AUA UAG AAG CCU C | |
| MM16 | 1 | GGA UCU UAU UUC UUC GGA G dTdT | Single U:U mismatch pair |
| | 10 | dTdT CCU UGA AUA AAG AAG CCU C | |
| MM17 | 1 | GGA UCU UAU UUC UUC GGA G dTdT | Single A:A mismatch pair |
| | 11 | dTdT CCA AGA AUA AAG AAG CCU C | |
| UB1 | 1 | GGA UCU UAU UUC UUC GGA G dTdT | May not exist as duplex at |
| | 12 | dTdT CCU Q₁₂GA AUA Q₁₂A Q₁₂ AAG Q₁₂CU C | physiological temperature |
| UB2 | 2 | GGA ACU UUU UUG UUC CGA G dTdT | One each of G:Q₁₂, C:Q₁₂, |
| | 12 | dTdT CCU Q₁₂GA AUA Q₁₂AQ₁₂ AAG Q₁₂CU C | U:Q₁₂ and A:Q₁₂ pairs |
| UB4 | 1 | GGA UCU UAU UUC UUC GGA G dTdT | Single G: Q₁₂ pair |
| | 13 | dTdT CCU AGA AUA AAG AAG Q12CU C | |
| UB8 | 1 | GGA UCU UAU UUC UUC GGA G dTdT | Single C: Q₁₂ pair |
| | 14 | dTdT CCU AGA AUA AA Q₁₂ AAG CCU C | |
| UB10 | 1 | GGA UCU UAU UUC UUC GGA G dTdT | Single U: Q₁₂ pair |
| | 15 | dTdT CCU AGA AUA Q₁₂AG AAG CCU C | |
| UB16 | 1 | GGA UCU UAU UUC UUC GGA G dTdT | Single U: Q₁₂ pair |
| | 16 | dTdT CCU Q₁₂GA AUA AAG AAG CCU C | |
| UB17 | 1 | GGA UCU UAU UUC UUC GGA G dTdT | Single A: Q₁₂ pair |
| | 17 | dTdT CCQ₁₂ AGA AUA AAG AAG CCU C | |
| 2M4 | 3 | GGA UCU UAU UUC UUC CGA GdTdT | |
| | 7 | dTdT CCU AGA AUA AAG AAG GCU C | |
| 2M8 | 4 | GGA UCU UAU UUG UUC GGA GdTdT | |
| | 8 | dTdT CCU AGA AUA AAC AAG CCU C | |
| 2M10 | 5 | GGA UCU UAU AUC UUC GGA GdTdT | |
| | 9 | dTdT CCU AGA AUA UAG AAG CCU C | |
| 2M16 | 37 | GGA ACU UAU UUC UUC GGA GdTdT | |
| | 10 | dTdT CCU UGA AUA AAG AAG CCU C | |

In Table 2 above, Q₁₂ indicates a 5-nitroindolyl (5-nitroindole).

TABLE 3
2,4-Difluorotoulyl (Q₁₀) and Inosine (I) comprising siRNA duplexes.

| Duplex | Seq ID | Modification | |
|---|---|---|---|
| 4F | 1 | GGA UCU UAU UUC UUC GGA G dTdT | One each of G:Q₁₀, C:Q₁₀, |
| | 19 | dTdT CCU Q₁₀GA AUA Q₁₀A Q₁₀ AAG Q₁₀CU C | U:Q₁₀ and A:Q₁₀ pairs |
| F4 | 1 | GGA UCU UAU UUC UUC GGA G dTdT | Single G: Q₁₀ pair |
| | 20 | dTdT CCU AGA AUA AAG AAG Q₁₀CU C | |
| F8 | 1 | GGA UCU UAU UUC UUC GGA G dTdT | Single C: Q₁₀ pair |
| | 21 | dTdT CCU AGA AUA AA Q₁₀ AAG CCU C | |
| F10 | 1 | GGA UCU UAU UUC UUC GGA G dTdT | Single U: Q₁₀ pair |
| | 22 | dTdT CCU AGA AUA Q₁₀AG AAG CCU C | |
| F16 | 1 | GGA UCU UAU UUC UUC GGA G dTdT | Single U: Q₁₀ pair |
| | 23 | dTdT CCU Q₁₀GA AUA AAG AAG CCU C | |
| F17 | 1 | GGA UCU UAU UUC UUC GGA G dTdT | Single A: Q₁₀ pair |
| | 24 | dTdT CCQ10 AGA AUA AAG AAG CCU C | |
| 7611(UC/GA) | 25 | CGA UCG UGC CUU CCU UUG AdT*dT | Positive control siRNA for |
| | 29 | dT*dTGCU AGC ACG GAA GGA AAC U | H1N1 strain |
| CU/AG | 33 | CGA UCG UGC CCU CUU UUG AdT*dT | Positive control siRNA for |
| | 34 | dT*dTGCU AGC ACG GGA GAA AAC U | H3N2 strain |
| CC/GG | 35 | CGA UCG UGC CCU CCU UUG AdT*dT | |
| | 36 | dT*dTGCU AGC ACG GGA GGA AAC U | |
| FF/AG | 26 | CGA UCG UGC CQ₁₀U CQ₁₀U UUG AdT*dT | |
| | 34 | dT*dTGCU AGC ACG GGA GAA AAC U | |
| FF/GA | 26 | CGA UCG UGC CQ₁₀U CQ₁₀U UUG AdT*dT | |
| | 29 | dT*dTGCU AGC ACG GAA GGA AAC U | |
| FC/GG | 27 | CGA UCG UGC CQ₁₀U CCU UUG AdT*dT | |
| | 36 | dT*dTGCU AGC ACG GGA GGA AAC U | |
| UC/II | 25 | CGA UCG UGC CUU CCU UUG AdT*dT | |
| | 32 | dT*dTGCU AGC ACG GIA GIA AAC U | |
| CU/II | 33 | CGA UCG UGC CCU CUU UUG AdT*dT | |
| | 32 | dT*dTGCU AGC ACG GIA GIA AAC U | |
| CC/II | 35 | CGA UCG UGC CCU CCU UUG AdT*dT | |
| | 32 | dT*dTGCU AGC ACG GIA GIA AAC U | |
| FF/II | 26 | CGA UCG UGC CQ₁₀U CQ₁₀U UUG AdT*dT | |
| | 32 | dT*dTGCU AGC ACG GIA GIA AAC U | |
| UC/FF | 25 | CGA UCG UGC CUU CCU UUG AdT*dT | |
| | 30 | dT*dTGCU AGC ACG GQ₁₀A GQ₁₀ A AAC U | |
| UC/GF | 25 | CGA UCG UGC CUU CCU UUG AdT*dT | |
| | 31 | dT*dTGCU AGC ACG GQ10A GGA AAC U | |
| CC/GF | 35 | CGA UCG UGC CCU CCU UUG AdT*dT | |
| | 31 | dT*dTGCU AGC ACG GQ₁₀A GGA AAC U | |
| II/FF | 28 | CGA UCG UGC CIU CIU UUG AdT*dT | |
| | 30 | dT*dTGCU AGC ACG GQ10A GQ₁₀ A AAC U | |

In Table 3 above, * indicates a phosphorothioate linkage; Q₁₀ indicates a 2,4-
difluorotoluyl (2,4-difluorotoluene); and I indicates inosine.

### Example 5

### UV Thermal Denaturation Studies

Molar extinction coefficients for the oligonucleotides were calculated according to nearest-neighbor approximations (units=10⁴ M⁻¹ cm⁻¹). Duplexes were prepared by mixing equimolar amounts of the complementary strands and lyophilizing the resulting mixture to dryness. The resulting pellet was dissolved in phosphate buffered saline (pH 7.0) to give a final concentration of 8 μM total duplex. The solutions were heated to 90° C. for 10 min and cooled slowly to room temperature before measurements. Prior to analysis, samples were degassed by placing them in a speed-vac concentrator for 2 min. Denaturation curves were acquired at 260 nm at a rate of heating of 0.5° C./min using a Varian CARY spectrophotometer fitted with a 12-sample thermostated cell block and a temperature controller. Results are shown in Table 4 below.

TABLE 4
Thermal stability of siRNA duplexes with A:X pair (X = U, A, G, C and Q₁₂).

| | | Tm ± 0.5 | ΔTm | |
|---|---|---|---|---|
| Duplex | Sequence | (° C.) | (° C.) | Remark |
| 1685 | GGA UCU UAU UUC UUC GGA G dTdT | 72.0 | 0.0 | Positive control siRNA |
| | dTdT CCU AGA AUA AAG AAG CCU C | | | |
| MM4 | GGA UCU UAU UUC UUC GGA G dTdT | 62.3 | −9.7 | Single G:G mismatch pair |
| | dTdT CCU AGA AUA AAG AAG GCU C | | | |
| MM8 | GGA UCU UAU UUC UUC GGA G dTdT | 59.8 | −12.2 | Single C:C mismatch pair |
| | dTdT CCU AGA AUA AAC AAG CCU C | | | |
| MM10 | GGA UCU UAU UUC UUC GGA G dTdT | 64.5 | −7.5 | Single U:U mismatch pair |
| | dTdT CCU AGA AUA UAG AAG CCU C | | | |
| MM16 | GGA UCU UAU UUC UUC GGA G dTdT | 63.8 | −8.2 | Single U:U mismatch pair |
| | dTdT CCU UGA AUA AAG AAG CCU C | | | |
| MM17 | GGA UCU UAU UUC UUC GGA G dTdT | 64.0 | −8.0 | Single A:A mismatch pair |
| | dTdT CCA AGA AUA AAG AAG CCU C | | | |
| UB1 | GGA UCU UAU UUC UUC GGA G dTdT | 33.5 | −38.5 | May not exist as duplex at |
| | dTdT CCU Q₁₂GA AUA Q₁₂A Q₁₂ AAG Q₁₂CU C | | | physiological temperature |
| UB4 | GGA UCU UAU UUC UUC GGA G dTdT | 61.6 | ₋₁₂.4 | Single G: Q₁₂ pair |
| | dTdT CCU AGA AUA AAG AAG Q₁₂CU C | | | |
| UB8 | GGA UCU UAU UUC UUC GGA G dTdT | 59.8 | ₋₁₂.2 | Single C: Q₁₂ pair |
| | dTdT CCU AGA AUA AA Q₁₂ AAG CCU C | | | |
| UB10 | GGA UCU UAU UUC UUC GGA G dTdT | 63.5 | −8.5 | Single U: Q₁₂ pair |
| | dTdT CCU AGA AUA Q₁₂AG AAG CCU C | | | |
| UB16 | GGA UCU UAU UUC UUC GGA G dTdT | 63.7 | −8.3 | Single U: Q₁₂ pair |
| | dTdT CCU Q₁₂GA AUA AAG AAG CCU C | | | |
| UB17 | GGA UCU UAU UUC UUC GGA G dTdT | 64.0 | −8.0 | Single A: Q₁₂ pair |
| | dTdT CCQ₁₂ AGA AUA AAG AAG CCU C | | | |

In Table 4 above, Q₁₂ indicates a 5-nitroindolyl (5-nitroindole).

### INCORPORATION BY REFERENCE

All of the patents and publications cited herein are hereby incorporated by reference.

### Equivalents

Those skilled in the art will recognize, or be able to ascertain using no more than routine experimentation, many equivalents to the specific embodiments of the invention described herein. Such equivalents are intended to be encompassed by the following claims.

We claim:

1. A method of reducing the expression level of a viral gene from two or more strains of a virus comprising
a. contacting a cell with a single oligonucleotide agent, said oligonucleotide agent comprising an antisense strand oligonucleotide consisting of 19 to 23 nucleotides in length and a sense strand oligonucleotide consisting of 19 to 23 nucleotides in length, wherein either or both strands comprises at least two universal bases located at two sites of sequence variability within a region of conservation among said two or more strains of a virus, wherein said antisense strand oligonucleotide is complementary with, and thereby hybridizes to, said region of conservation among said two or more strains of a virus, and
b. measuring the RNA or protein levels of said two or more strains of the virus in said cells, where a reduction in either RNA or protein levels measured demonstrates the oligonucleotide agent has reduced the expression level of multiple strains of a virus.

2. The method of claim 1 wherein at least 19 nucleotides of said sense strand oligonucleotide are complementary to said antisense strand oligonucleotide producing a double-stranded oligonucleotide agent comprising a single strand or unpaired region at one or both ends.

3. The method of claim 1 wherein the virus is the influenza virus.

4. The method of claim 3 wherein the at least two strains are the H1N1 and the H3N2 strains of influenza.

5. The method of claim 1 wherein the cells are MDCK or Vero cells.

6. The method of claim 1 wherein expression levels are determined by the ELISA method.

7. The method of claim 1 wherein the antisense strand oligonucleotide comprises exactly two universal nucleobases.

8. The method of claim 1 wherein only the antisense strand oligonucleotide comprises exactly two universal nucleobases.

9. The method of claim 8, wherein said exactly two universal nucleobases are located at positions 6 and 9 as counted from the 5′ end of the antisense strand oligonucleotide.

10. The method of claim 9 wherein at least one of said universal nucleobases is 2,4-difluorotoluyl.

11. The method of claim 10, wherein both of said universal nucleobases is 2,4-difluorotoluyl.

12. A method of overcoming viral target degeneracy when targeting viral genes comprising the steps of
a. identifying a region of conservation among two or more strains of a virus, wherein said region is at least 21 nucleotides in length and has no more than two sites of sequence variability;
b. selecting an oligonucleotide agent 19-23 nucleotides in length that is complementary to said region of conservation; and
c. synthesizing said oligonucleotide agent selected in step (b) wherein said oligonucleotide agent comprises a universal nucleobase at one or both sites of sequence variability thereby producing a modified oligonucleotide agent which is less selective in pairing with juxtaposing natural nucleobases.

13. The method of claim 12 further comprising
a. contacting a cell with a single oligonucleotide agent, said oligonucleotide agent comprising an antisense strand oligonucleotide consisting of 19 to 23 nucleotides in length and a sense strand oligonucleotide consisting of 19 to 23 nucleotides in length, wherein either or both strands comprises at least two universal bases located at two sites of sequence variability within a region of conservation among said two or more strains of a virus, wherein said antisense strand oligonucleotide is complementary with, and thereby hybridizes to, said region of conservation among said two or more strains of a virus, and
b. measuring the RNA or protein levels of said two or more strains of the virus in said cells, where a reduction in either RNA or protein levels measured demonstrates the oligonucleotide agent has reduced the expression level of multiple strains of a virus.

14. The method of claim 12 wherein said oligonucleotide agent consisting of an antisense oligonucleotide strand consisting of 19 to 23 nucleotides in length and a sense strand oligonucleotide consisting of 19 to 23 nucleotides in length and wherein at least 19 nucleotides of said sense strand oligonucleotide are complementary to said antisense strand oligonucleotide producing a double-stranded oligonucleotide agent comprising a single strand or unpaired region at one or both ends.

15. The method of claim 13 wherein the virus is the influenza virus.

16. The method of claim 15 wherein the at least two strains are the H1N1 and the H3N2 strains of influenza.

17. The method of claim 15 wherein expression levels are determined by the ELISA method.

18. The method of claim 14 wherein the antisense strand oligonucleotide comprises exactly two universal nucleobases.

19. The method of claim 14 wherein only the antisense strand oligonucleotide comprises exactly two universal nucleobases.

20. The method of claim 19, wherein said exactly two universal nucleobases are located at positions 6 and 9 as counted from the 5′ end of the antisense strand oligonucleotide.

21. The method of claim 20 wherein at least one of said universal nucleobases is 2,4-difluorotoluyl.

22. The method of claim 21, wherein both of said universal nucleobases is 2,4-difluorotoluyl.


SEQUENCE LISTING (37 sequences)

SEQ ID NO: 1 - DNA, 21 bp, Artificial Sequence
  Description of Combined DNA/RNA Molecule Synthetic oligonucleotide
ggaucuuauuucuucggagtt

SEQ ID NO: 2 - DNA, 21 bp, Artificial Sequence
  Description of Combined DNA/RNA Molecule Synthetic oligonucleotide
ggaacuuuuuuguuccgagtt

SEQ ID NO: 3 - DNA, 21 bp, Artificial Sequence
  Description of Combined DNA/RNA Molecule Synthetic oligonucleotide
ggaucuuauuucuuccgagtt

SEQ ID NO: 4 - DNA, 21 bp, Artificial Sequence
  Description of Combined DNA/RNA Molecule Synthetic oligonucleotide
ggaucuuauuuguucggagtt

SEQ ID NO: 5 - DNA, 21 bp, Artificial Sequence
  Description of Combined DNA/RNA Molecule Synthetic oligonucleotide
ggaucuuauaucuucggagtt

SEQ ID NO: 6 - DNA, 21 bp, Artificial Sequence
  Description of Combined DNA/RNA Molecule Synthetic oligonucleotide
cuccgaagaaauaagaucctt

SEQ ID NO: 7 - DNA, 21 bp, Artificial Sequence
  Description of Combined DNA/RNA Molecule Synthetic oligonucleotide
cucggaagaaauaagaucctt

SEQ ID NO: 8 - DNA, 21 bp, Artificial Sequence
  Description of Combined DNA/RNA Molecule Synthetic oligonucleotide
cuccgaacaaauaagaucctt

SEQ ID NO: 9 - DNA, 21 bp, Artificial Sequence
  Description of Combined DNA/RNA Molecule Synthetic oligonucleotide
cuccgaagauauaagaucctt

SEQ ID NO: 10 - DNA, 21 bp, Artificial Sequence
  Description of Combined DNA/RNA Molecule Synthetic oligonucleotide
cuccgaagaaauaaguucctt

SEQ ID NO: 11 - DNA, 21 bp, Artificial Sequence
  Description of Combined DNA/RNA Molecule Synthetic oligonucleotide
cuccgaagaaauaagaacctt

SEQ ID NO: 12 - DNA, 21 bp, Artificial Sequence
  Description of Combined DNA/RNA Molecule Synthetic oligonucleotide
cucngaananauaagnucctt

SEQ ID NO: 13 - DNA, 21 bp, Artificial Sequence
  Description of Combined DNA/RNA Molecule Synthetic oligonucleotide
cucngaagaaauaagaucctt

SEQ ID NO: 14 - DNA, 21 bp, Artificial Sequence
  Description of Combined DNA/RNA Molecule Synthetic oligonucleotide
cuccgaanaaauaagaucctt

SEQ ID NO: 15 - DNA, 21 bp, Artificial Sequence
  Description of Combined DNA/RNA Molecule Synthetic oligonucleotide
cuccgaaganauaagaucctt

SEQ ID NO: 16 - DNA, 21 bp, Artificial Sequence
  Description of Combined DNA/RNA Molecule Synthetic oligonucleotide
cuccgaagaaauaagnucctt

SEQ ID NO: 17 - DNA, 21 bp, Artificial Sequence
  Description of Combined DNA/RNA Molecule Synthetic oligonucleotide
cuccgaagaaauaagancctt

SEQ ID NO: 18 - DNA, 21 bp, Artificial Sequence
  Description of Combined DNA/RNA Molecule Synthetic oligonucleotide
cucugaacauauaaguucctt

SEQ ID NO: 19 - DNA, 21 bp, Artificial Sequence
  Description of Combined DNA/RNA Molecule Synthetic oligonucleotide
cucngaananauaagnucctt

SEQ ID NO: 20 - DNA, 21 bp, Artificial Sequence
  Description of Combined DNA/RNA Molecule Synthetic oligonucleotide
cucngaagaaauaagaucctt

SEQ ID NO: 21 - DNA, 21 bp, Artificial Sequence
  Description of Combined DNA/RNA Molecule Synthetic oligonucleotide
cuccgaanaaauaagaucctt

SEQ ID NO: 22 - DNA, 21 bp, Artificial Sequence
  Description of Combined DNA/RNA Molecule Synthetic oligonucleotide
cuccgaaganauaagaucctt

SEQ ID NO: 23 - DNA, 21 bp, Artificial Sequence
  Description of Combined DNA/RNA Molecule Synthetic oligonucleotide
cuccgaagaaauaagnucctt

SEQ ID NO: 24 - DNA, 21 bp, Artificial Sequence
  Description of Combined DNA/RNA Molecule Synthetic oligonucleotide
cuccgaagaaauaagancctt

SEQ ID NO: 25 - DNA, 21 bp, Artificial Sequence
  Description of Combined DNA/RNA Molecule Synthetic oligonucleotide
cgaucgugccuuccuuugatt

SEQ ID NO: 26 - DNA, 21 bp, Artificial Sequence
  Description of Combined DNA/RNA Molecule Synthetic oligonucleotide
cgaucgugccnucnuuugatt

SEQ ID NO: 27 - DNA, 21 bp, Artificial Sequence
  Description of Combined DNA/RNA Molecule Synthetic oligonucleotide
cgaucgugccnuccuuugatt

SEQ ID NO: 28 - DNA, 21 bp, Artificial Sequence
  Description of Combined DNA/RNA Molecule Synthetic oligonucleotide
cgaucgugccnucnuuugatt

SEQ ID NO: 29 - DNA, 21 bp, Artificial Sequence
  Description of Combined DNA/RNA Molecule Synthetic oligonucleotide
ucaaaggaaggcacgaucgtt

SEQ ID NO: 30 - DNA, 20 bp, Artificial Sequence
  Description of Combined DNA/RNA Molecule Synthetic oligonucleotide
ucaaanganggcacgaugtt

SEQ ID NO: 31 - DNA, 21 bp, Artificial Sequence
  Description of Combined DNA/RNA Molecule Synthetic oligonucleotide
ucaaagganggcacgaucgtt

SEQ ID NO: 32 - DNA, 21 bp, Artificial Sequence
  Description of Combined DNA/RNA Molecule Synthetic oligonucleotide
ucaaanganggcacgaucgtt

SEQ ID NO: 33 - DNA, 21 bp, Artificial Sequence
  Description of Combined DNA/RNA Molecule Synthetic oligonucleotide
cgaucgugcccucuuuugatt

SEQ ID NO: 34 - DNA, 21 bp, Artificial Sequence
  Description of Combined DNA/RNA Molecule Synthetic oligonucleotide
ucaaaagagggcacgaucgtt

SEQ ID NO: 35 - DNA, 21 bp, Artificial Sequence
  Description of Combined DNA/RNA Molecule Synthetic oligonucleotide
cgaucgugcccuccuuugatt

SEQ ID NO: 36 - DNA, 21 bp, Artificial Sequence
  Description of Combined DNA/RNA Molecule Synthetic oligonucleotide
ucaaaggagggcacgaucgtt

SEQ ID NO: 37 - DNA, 21 bp, Artificial Sequence
  Description of Combined DNA/RNA Molecule Synthetic oligonucleotide
ggaacuuauuucuucggagtt